(19) United States
(12) Patent Application Publication
(10) Pub. No.: US 2015/0306128 A1
(43) Pub. Date: Oct. 29, 2015

(54) COBALAMINS TO HELP MAINTAIN A NORMAL BODY WEIGHT AND A HEALTHY BODY MASS INDEX (BMI) IN UNDERWEIGHT, OVERWEIGHT AND OBESE HUMANS

(71) Applicant: Ernest Timothy Armstrong, Palm Desert, CA (US)

(72) Inventor: Ernest Timothy Armstrong, Palm Desert, CA (US)

(21) Appl. No.: 14/260,191

(22) Filed: Apr. 23, 2014

(51) Int. Cl. A61K 31/714; A61K 31/525; A61K 31/122; A61K 33/06; A61K 31/205

(52) U.S. Cl. CPC A61K 31/714; A61K 33/06; A61K 31/205; A61K 31/122; A61K 31/525

(57) ABSTRACT

The current invention discloses novel uses of orally ingested cobalamins for the maintenance of a healthy and normal body weight and body mass index (BMI). Cobalamins support mitochondrial conversion of carbohydrates into chemical energy called ATP. Cells in the body, especially energy-hungry muscle and brain cells, use ATP as an energy source for many types of cellular functions. Simply put, cobalamins are the natural nutrients needed for cellular metabolism and the burning of calories. In a phase III, placebo-controlled, nutritional study with a total of 1,551 human test subjects, on average those obese people who took cobalamins lost weight and BMI, while those obese people who took placebo gained weight and BMI.

### FIELD OF THE INVENTION

The current invention discloses a nutritional product (cobalamin) to support normal weight in humans and animals who are underweight, overweight or obese. In one embodiment, the current invention is ingested by humans as a cobalamin-containing tablet that is dissolved in the mouth and then swallowed.

### BACKGROUND OF THE INVENTION

The inventor of the current patent designed and directed studies on 1,551 human test subjects. The surprising, unexpected and serendipitous discoveries from that study relate the current invention.

Methods: Twenty three investigators administered orally-dissolving tablets with 3.3 mg cobalamins twice daily for 42 days to 1,551 test subjects with ragweed allergy, ages 12 to 75. Both before and after the use of cobalamins, the test subjects were given a physical exam that included documentation of their height and weight. The test subjects were not instructed to engage in special exercise or to follow any calorie-restricted diet as part of the study protocol. During the study, the test subjects were not permitted to take additional supplements containing cobalamins or folic acid. The study protocols were originally designed to evaluate the effectiveness of cobalamins on seasonal allergic rhinitis (i.e. hay fever), and as such was not designed as a weight-control study.

Results: The following unexpected discoveries were made when the pre-study physical exam results were compared to the post-study physical exam results. On average, the body weights and body mass indices (BMI) for 1.) the underweight people increased in the active group but decreased in the placebo group, 2.) the overweight people essentially remained the same in the active group but increased the placebo group, and 3.) the obese people decreased in the active group (n=224) but increased in the placebo group (n=205).

It appears from the study results, that the group of test subjects who were the most in need of losing weight, (that is the obese group), was the group that benefited most from the use of cobalamins.

Less than one percent of the test subjects in the studies were cobalamin deficient prior to their first blood draw (that is prior to their use of cobalamins in the study). Therefore, the current invention can be used by humans who are deficient in cobalamins or who are not deficient.

Cobalamin can be used to support normal weight in humans and animals that are underweight, overweight or obese by itself or in combination with other weight gain or weight loss agents, diets or exercise programs.

### DETAILED DESCRIPTION OF THE PRODUCT

One especially preferred embodiment of the current invention is a once- or twice-daily dissolving tablet that is ingested by placing in the mouth where it dissolves and then is swallowed; and contains combinations of cyanocobalamin, methylcobalamin, and adenosylcobalamin in amounts that provide healthy support to the mitochondria and cells. The current invention may also include one or more of the following substances: magnesium, coenzyme Q10, L-carnitine, and riboflavin.

Cobalamin is a general term for the various analogues of vitamin B12. Other terms for vitamin B12 include B12, B-12, vitamin B12 and vitamin B12.

Cyanocobalamin contains a —CN (cyano) group attached to the cobalt. Once inside the body, the —CN generally disassociates itself from the cobalt, and the resulting molecule picks up a methyl —CH3 on the cobalt, thus becoming methylcobalamin. These are also known as cyano-B12 and methyl-B12. Cyanocobalamin and methylcobalamin are commonly sold as supplements. Other analogues are possible, and are often casually referred to as cobalamins or vitamin B12.

Cyanocobalamin is a synthetic form of vitamin B12. The chemical name is 5,6-dimethylbenzimidazolyl cyanocobamide. The cobalt content is 4.35%. The molecular formula is C₆₃H₈₈CoN₁₄O₁₄P, which corresponds to a molecular weight of 1355.38.

These analogues of cobalamin include but are not limited to: adenosylcobalamin, cyanocobalamin, methylcobalamin, hydroxocobalamin, and hydroxycobalamin; the structures of these various forms include an adenosyl, CN, CH.sub.3, OH, and OH. Herein, the terms cobalamin, Cbl, vitamin B12, vit, B12, vitamin B.sub.12 and B12 can be used interchangeably with these molecules. The fundamental ring system without cobalt (Co) or side chains is called corrin and the octadehydrocorrin is called corrole. The corrin ring has attached six amidoalkyl (H.sub.2NC(O)Alk) substituents, at the 2, 3, 7, 8, 13 and 18 positions, which can be designated a-e and g, respectively. See D. L. Anton et al., J. Amer. Chem. Soc., 102, 2215 (1980).

As a substance, cobalamin can be utilized in its native form, or chemically altered into related chemical entities and derivatives such as metabolites, acids, hydroxyl groups, esters, salts, alcohols, acyl groups, and so forth in ways which would be obvious to one skilled in the art.

Each of the analogues of vitamin B12 (including but not limited to adenosylcobalamin, cyanocobalamin, methylcobalamin, hydroxocobalamin, hydroxycobalamin, aquacobalamin, and nitrocobalamin) can be used separately in the current invention, or in combination with other analogues.

The cyanocobalamin is produced by fermentation using a micro-organism.

Cyanocobalamin (also known as CNCbl, or 5,6-dimethylbenzimidazolyl cyanocobamide) has the molecular formula C63H88CoN14O14P. Cyanocobalamin is a manufactured commercial form of a cobalamin, and not native to the human body. Once inside the body cyanocobalamin is converted to methylcobalamin and adenosylcobalamin, but not to hydroxocobalamin.

Methylcobalamin (also known as mecobalamin, or MeCbl) has the molecular formula C63H91CoN13O14P and is notable as a rare example of an enzyme that contains metal-alkyl bonds. Methylation is the donation of a methyl group to a substrate, and methylcobalamin can function as the donor molecule. Proper DNA replication and cell division require methylation. For this reason, and others, the current invention includes cyanocobalamin and methylcobalamin.

Adenosylcobalamin (also known as cobamamide, AdCbl, or dibencozide) comprises more than 70 percent of the cobalamins in the brain and contains the word “Obama”. Adenosylcobalamin functions in reactions in which hydrogen groups and organic groups exchange places. Adenosylcobalamin is the major form in cellular tissues, especially energy-hungry muscles, where it is retained in the mitochondria. Adenosylcobalamin is the coenzyme for the mitochondrial enzyme methylmalonyl CoA mutase. Problems with methylmalonyl CoA mutase can lead to methylmalonic aciduria and dysfunction of the mitochondria. In one preferred embodiment of the current invention, adenosylcobalamin is included to support the normal function of the mitochondria.

Magnesium ions are important to the production of nucleic acid, DNA, and RNA, and the catalytic action of many enzymes. Of special relevance to the current invention are the magnesium-dependent enzymes associated with the conversion of adenosine triphosphate (ATP) into adenosine diphosphate (ADP) in the mitochondria. Phosporylation is an important process that occurs in the mitochondria. For this reason, one particularly preferred embodiment of the current invention includes elemental magnesium, magnesium oxide, magnesium gluconate, magnesium citrate, magnesium oxide, magnesium orotate, magnesium malate, and combinations thereof in the formulation in amounts ranging from about 10 mg to about 500 mg per portion.

Proper functioning of the mitochondria requires coenzyme Q10 (CoQ10), also known as ubiquinone or 1-4-benzoquinone. In one preferred embodiment, coenzyme Q10 is included in the formulation in amounts ranging from about 10 mg to about 500 mg per portion.

Riboflavin (vitamin B2) has an important function in energy metabolism. Flavin mononucleotide (FMN) and flavin adenine dinucleotide (FAD) function as coenzymes for a wide variety of oxidative enzymes and remain bound to the enzymes during the oxidation-reduction reactions. Reduction of isoalloxazine ring (FAD, FMN oxidized form) yields the reduced forms of the flavoproteins (FMNH2 and FADH2). For this reason, one particularly preferred embodiment of the current invention includes riboflavin in the formulation in amounts ranging from about 0.1 mg to about 300 mg per portion.

Levocarnitine (or L-carnitine) plays an important role in energy metabolism by helping the transport of fatty acids from the cytosol into the mitochondria. Also, it helps remove toxic chemical byproducts from the mitochondria so they do not accumulate. In one preferred embodiment of the current invention, L-carnitine, acetyl-L-carnitine (L-acetylcarnitine), L-propionyl carnitine, or L-carnitine fumarate, and combinations thereof is included in doses between 1 mg and 400 mg per portion.

In the current invention, formulation of a dissolving tablet can employ hydrophilic polymers that rapidly dissolve in the mouth, preferably on top of the tongue. The cyanocobalamin, methylcobalamin, and adenosylcobalamin permeate the skin of the mouth and, in a certain percentage, are ingested for absorption by the gut, especially the ileum. In one preferred embodiment of the current invention, formulation of a dissolving tablet involves the application of both aesthetic and performance characteristics such as polymers, plasticizers, active pharmaceutical ingredients, sweetening agents, saliva stimulating agents, flavoring agents, coloring agents, stabilizing and thickening agents. In the current invention, formulation of a dissolving tablet can employ polymers such as maltodextrin, microcrystalline cellulose and piroxicam made with a hot extrusion technique. To make the tablet more flexible; plasticizer excipients such as propylene glycol, glycerol, dimethyl phthalate, diethyl phthalate, dibutyl phthalate, triacrtin, castor oil, triethyl citrate, tributyl citrate, acetyl citrate in the current invention. In one preferred embodiment of the current invention Stevia (steviol glycoside) is used to sweeten the tablets.

A list of delivery vehicles includes, which is not meant to limit the scope of the invention; liquid, drops, dots, elixirs, solutions, suspensions, powders, spray powders, tablets, capsules, lozenges, nasal sprays, film strips, syrups, creams, ointments and suppositories. Each of said delivery vehicles can have excipients, binding agents, penetration enhancers, sweeteners and other inactive ingredients. Cobalamin described in the current invention can be administered to humans or other animals via delivery means that include, but are not limited to; oral, transdermal, intranasal, transmucosal, buccal, injectable, parenteral and inhaled. Other delivery means are possible to one skilled in the art.

In one embodiment, the product is delivered to in a dissolving filmstrip placed in the mouth. The dissolving product is a thin film delivery technology, and is also referred to as a dissolving film or an oral strip.

The current invention can be placed in the feed or water of domestic animals, poultry, pigs, cattle, sheep and other livestock for weight normalization.

When considering the various embodiments of the invention described herein, those knowledgeable in the art will appreciate that these are illustrative only. Such embodiments do not limit the scope of the invention. Those knowledgeable in the art involved will appreciate that many variations, substitutions, equivalents, and like modifications may be made within the scope of the present invention.

Following are the basic metabolitic actions of cobalamins, and their possible mechanisms of action in weight normalization. Reactions catalyzed by cobalamin dependent enzymes occur in the mitochondria. It is possible, that this pathway through the mitochondria and its effects on one's ability to convert chemical energy into muscle movement, is responsible for the reports that cobalamin makes one feel energetic and increases one's “energy”, thus resulting in more physical activity, calorie expenditures and weight loss.

Most of the body's energy is chemical energy manufactured in the mitochondria and stored in the form of ATP. Mitochondria live as organelles within all cells. The number of mitochondria per cell can range from one to thousands, depending on the energy needs of the cell. Energy-hungry muscle and brain cells contain thousands of mitochondria. Once inside the body cyanocobalamin is converted to adenosylcobalamin and methylcobalamin. Adenosylcobalamin and methylcobalamin are especially critical to the health and functioning of the rest of the mitochondria.

The mitochondrion (plural mitochondria) is the “cell's powerhouse”. Most of the organism's stored energy is converted into a usable chemical energy known as adenosine triphosphate (ATP) in the mitochondria. The citric acid cycle or Krebs cycle generates GTP which becomes ATP. Problems with the mitochondria can cause them to die. Problems with the mitochondria, which are also involved in cell signaling, cell death, and cell differentiation, can disrupt the functioning of the cell, tissue and organ in which they survive. It is an organelle with its own strand of DNA, distinct from DNA in the nucleus. Mitochondria are found inside most animal cells. Populations of mitochondria per cell range from one to thousands. Mitochondria living in our cells may be hitch-hiking, symbiotic descendants of bacteria that provided some benefits to us, indeed mitochondrial DNA resembles bacterial DNA. We certainly provided a safe living cell as home with all the warmth and nutrients to these bacteria. When one realizes that the basic chemical structure of cobalamins can only be synthesized by bacteria, it is not hard to see a critical connection and history between mitochondria and cobalamins.

Cobalamin is a water-soluble vitamin with an important role in biochemical processes referred to as single carbon transfers. During these reactions, functional units such as methyl groups (—CH3) are transferred onto or between biologically important compounds. Cobalamin is a co-factor for at least three enzymes that carry out these types of reactions, acting as a transitional carrier of the single carbon group. Cobalamin catalyzes methionine synthase.

Cobalamin and methionine play a regulatory role in the synthesis of nucleotides, which are DNA precursors. Cobalamin deficiency may cause a defect in thymidylate synthesis, leading to derangement of DNA synthesis. Given that cobalamin is necessary for DNA synthesis, affecting essentially all mammalian cell replication, there are many possible ways in which cobalamin may affect weight control, including but not limited to: cell growth and replication in the nervous system (such as weight and appetite control centers in the brain), cardiovascular system, muscular system (increased growth of muscle tissue), bone (replication of osteocytes), immune system disorders, digestion and absorption of food, and others obvious to those familiar with the art.

### DETAILED DESCRIPTION OF THE ORIGINAL RESEARCH

### Example 1

### Ragweed Allergy Study Yielded Surprising Results in Regard to Weight Control

Methods: From early August to early November 2006 test subjects with demonstrated allergy to ragweed pollen participated in twin studies titled: A Phase 3, randomized, double-blind, placebo-controlled, parallel group study of the safety and efficacy of pre-seasonal sublingual cyanocobalamin orally-dissolving tablets on moderate to moderately severe seasonal allergic rhinitis in humans. (There were identical, twin studies under protocol numbers RA3333 and RA5555, and under FDA IND 68,994.)

Test subjects were randomized into 2 study arms and given Cyanocobalamin, USP delivered in an orally-dissolving tablet 3.3 mg per tablet twice daily for 42 consecutive days or placebo.

Informed consent was given. Test subjects were given physical exams and gave blood/urine samples for safety before and after the use of cobalamins or placebo at one of 23 immunology clinics in the Midwest, Texas, Northeast, and South, all regions of the USA. Essentially all of the investigators were Board Certified in Allergy/Immunology.

Case report forms (CRF) were kept, and the study was properly monitored. Any report of an adverse event (AE) was documented in a diary and transcribed to the appropriate CRF page.

Both before and after the use of cobalamins or placebo, test subjects were given a physical exam that included documentation of their height and weight. The test subjects were not instructed to engage in special exercise or to follow any restricted-calorie diet as part of the study regimen. During the study, the test subjects were not permitted to take additional supplements containing B12 or folic acid. The study protocols were originally designed to evaluate the effectiveness of cobalamins on seasonal allergic rhinitis (i.e. hay fever), and as such was not designed as a weight-loss study.

This nutritional study was designed and directed by the inventor of the current patent. A large, multi-center, Phase 3, randomized, placebo-controlled nutritional study on 1,551 test subjects was designed and directed by the inventor of the current patent. Methods: The study was titled: “A Phase 3, randomized, double-blind, placebo-controlled, parallel group study of the safety and efficacy of pre-seasonal cyanocobalamin orally-dissolving tablets on moderate to moderately severe seasonal allergic rhinitis in humans”. The study took place before and during the ragweed pollen season at 23 sites in the Midwest, Northeast and Central Texas regions of the United States. Essentially all of the 23 investigators were Board Certified in Allergy/Immunology. Qualified test subjects were randomized into an active or placebo group (approximately 50% and 50%) using an interactive voice recognition system (IVRS). All subjects (or their guardians) signed an Informed Consent form approved by the IRB. Each subject had three visits to the clinic. At Visit 1 and at Visit 3, they were given a physical exam (HEENT, chest, lungs, heart, vital signs, height and weight); and donated blood and urine samples for laboratory analysis. CBC and chemistry panels were run for safety analyses. The blood samples were analyzed by chemiluminescent immunoassay for the presence of ragweed specific immunoglobulin epsilon (IgE), and were assayed for cobalamins (cyanocobalamin, methylcobalamin and adenosylcobalamin) levels.

Test subjects self-rated the severity their allergy symptoms in the morning (a.m.) and in the evening (p.m.) by entering a numeric score in a keypad of a telephone (IVRS) or in a computer connected via the Internet to a database.

Subjects were given their study product in the a.m. and p.m.: either 3.3 mg Cyanocobalamin, USP or placebo. Subjects were instructed to let the study product “dissolve completely in your mouth, especially under your tongue, then swallow.”. Subjects self-administered the study products for six consecutive weeks. For the next four weeks subjects did not take any study products.

Any adverse event (AE) or serious adverse event (SAE) was documented by the subject in a paper diary and then transcribed to the appropriate case report form (CRF) page. All SAES were attended to by the investigator, and reported to the FDA by the sponsor. All sites were monitored multiple times by qualified monitors.

Results: There was a total enrollment of 1,551 subjects (RA5555 n=763 and RA3333 n=788). The total number of doses possible was 84 doses. Over 50 percent (n=766) of the 1474 subjects who reported taking at least one dose, took at least 80 doses of cyanocobalamin or placebo.

The allergy symptom scores were derived by summing and averaging all a.m. plus all p.m. scores for the symptoms of sneezing, runny nose, nasal congestion, nasal itch and eye itch. The primary comparison of interest was the scores across Weeks 4, 5 and 6 (i.e. during the pollen season). The reduction in symptom severity from baseline was greater for the active group than the placebo group for all five composite symptoms: sneezing, runny nose, nasal congestion, nasal itch and eye itch.

In terms of safety, the active study product was very well tolerated.

As per the laboratory results, a significant average increase of more than 250 percent in post-use-of-cobalamin blood serum cobalamin (cyanocobalamin, methylcobalamin and adenosylcobalamin) levels was reported in the cyanocobalamin-administered subject groups compared with essentially no increase in placebo-administered subjects.

Less than one percent of the test subjects in the studies were cobalamin deficient prior to their first blood draw (i.e. prior to their use of cobalamins in the study).

The following surprising and serendipitous discoveries were made when the pre-study physical exam results were compared to the post-study physical exam results.

The body weights and in body mass indices (BMI) changed as follows: 1.) For the underweight people, on average the active group (n=14) increased and the placebo group (n=24) decreased, 2.) For the overweight people, on average the active group (n=191) essentially remained the same and the placebo group (n=214) increased, and 3.) For the obese people, on average the active group (n=224) decreased and the placebo group (n=205) increased.

The body mass index (BMI), or Quetelet index, is a measure of relative weight based on an individual's mass and height. The following parameters are used: “Underweight” is defined as a BMI of under 18.50, “Normal” is defined as a BMI of between 18.50 and 24.99, “Overweight” is defined as a BMI of between 25.00 and 29.99, and “Obese” is defined as a BMI of 30.00 and over.

Change in body mass index (BMI) for each test subject, with the pre-cobalamin/placebo use physical exam (Visit 2) compared to the post-cobalamin/placebo use physical exam (Visit 3). The following data are from the twin ragweed allergy studies (protocol numbers RA3333 and RA5555). The data are the average (mean) changes in body mass index from the first physical exam. (n=means the number of test subjects observed, and “Active” means those receiving cobalamins.)

Underweight Test Subjects Mean Change in BMI
Protocol No. RA3333: 0.01 Active (n=2), and −0.65 Placebo (n=10)
Protocol No. RA5555: 0.14 Active (n=12), and 0.08 Placebo (n=14)

Normal Test Subjects Mean Change in BMI
Protocol No. RA3333: −0.04 Active (n=123), and −0.02 Placebo (n=106)
Protocol No. RA5555: −0.04 Active (n=118), and −0.11 Placebo (n=112)

Overweight Test Subjects Mean Change in BMI
Protocol No. RA3333: 0.07 Active (n=84), and 0.13 Placebo (n=99)
Protocol No. RA5555: 0.05 Active (n=107), and 0.11 Placebo (n=115)

Obese Test Subjects Mean Change in BMI
Protocol No. RA3333: −0.14 Active (n=105), and 0.20 Placebo (n=89)
Protocol No. RA5555: 0.01 Active (n=119), and 0.13 Placebo (n=116)

### DETAILED DESCRIPTION OF THE PREFERRED EMBODIMENTS

One especially preferred embodiment of the current invention is a once-daily dissolving tablet that is placed in the mouth and swallowed, and contains combinations of cyanocobalamin, methylcobalamin, adenosylcobalamin, magnesium, coenzyme Q10, and riboflavin in amounts that provide support to the mitochondria and cells.

The current invention was reduced to practice and successfully tested in humans in a variety of formulas.

The manufacturing and packaging procedures to make the orally-dissolving tablet are: 1) Weigh each ingredient into individual containers, 2) If any of the ingredients appear to be agglomerated, pass that ingredient through an appropriate mesh screen, 3) Place the sorbitol, the silica, the cherry powder, and the cyanocobalamin (and if used, the methylcobalamin and adenosylcobalamin) into an appropriate blender, 4) Mix until the blend appears to be uniform in color, 5) Add the fructose to the blender, 6) Mix until uniform, 7) Pass the stearic acid (and the magnesium stearate and if used, the riboflavin, L-carnitine and coenzyme 10) through an appropriate mesh screen, 8) Add the screened ingredients to the blender, 9) Mix for 3 minutes, 10) Remove blend, 11) Compress blend into tablets using an appropriate tablet press fitted with the appropriate tablet punches, 12) Collect tablets into bulk containers,13) Package into appropriate, light-blocking container—closure systems, and, if appropriate, add a cotton or rayon ball and a desiccant.

Formula 1 is an especially preferred embodiment because it is the formula that was used in the 1,551 person study, with each orally-dissolving tablet containing these ingredients in the approximate amounts: cyanocobalamin 3.3 mg, sorbitol, NF, 285 mg, fructose 285 mg, cherry powder 25 mg, stearic acid NF 20 mg, and silica NF 6 mg.

Formula 2 is an especially preferred embodiment because it is the formula that was manufactured and tested, with each orally-dissolving tablet containing these ingredients: about 3.34 mg cyanocobalamin, about 0.214 mg methylcobalamin, from about 0.05 mg to about 0.13 mg adenosylcobalamin, about 3 mg magnesium, about 0.1 mg L-carnitine, about 5 mg coenzyme Q10, about 3.06 mg riboflavin, about 250 mg xlyitol, about 78 mg glycine, about 62.22 mg inositol, about 50 mg cherry flavor powder, about 28 mg taurine, about 10 mg microcrystalline cellulose, about 5 mg sorbitol, about 1.01 mg Syloid 244, and 1 mg lauric acid. Most of the above ingredients have an extra overage so that a long shelf life can be assured.

Formula 3 was a dissolving tablet with 3.3 mg of cyanocobalamin.

Formula 4 was a dissolving tablet with 6.6 mg of cyanocobalamin.

Formula 5 was a dissolving tablet with 3.3 mg of methylcobalamin.

Formula 6 was a dissolving tablet with 3.3 mg of adenosylcobalamin.

Formula 7 was a dissolving tablet with 2.2 mg of cyanocobalamin, 2.2 mg of methylcobalamin, and 2.2 mg of adenosylcobalamin.

Formula 8 was a dissolving tablet with 3.3 mg of adenosylcobalamin.

Formula 9 was a dissolving tablet with 5.6 mg of cyanocobalamin, 0.5 mg of methylcobalamin, and 0.5 mg of adenosylcobalamin.

Formula 10 was a dissolving tablet with 1.1 mg of cyanocobalamin, 1.1 mg of methylcobalamin, and 1.1 mg of adenosylcobalamin.

Formula 11 was a dissolving tablet with 2.2 mg of cyanocobalamin, 2.2 mg of methylcobalamin, 2.2 mg of adenosylcobalamin, 15 mg of coenzyme Q10, and 2.1 mg of riboflavin.

Formula 12 was a dissolving tablet with 1.1 mg of cyanocobalamin, 1.1 mg of methylcobalamin, 1.1 mg of adenosylcobalamin, 18 mg of coenzyme Q10, and 2.1 mg of riboflavin.

Formula 13 was a dissolving tablet with 1.1 mg of cyanocobalamin, 1.1 mg of methylcobalamin, 1.1 mg of adenosylcobalamin, 5 mg magnesium, 9 mg of coenzyme Q10, 5 mg L-carnitine, and 2.1 mg of riboflavin.

Formula 14 was a dissolving tablet with 5.6 mg of cyanocobalamin, 0.5 mg of methylcobalamin, 0.5 mg of adenosylcobalamin, 15 mg of coenzyme Q10, and 1 mg of riboflavin.

Formula 15 was a dissolving tablet with 5.6 mg of cyanocobalamin, 0.5 mg of methylcobalamin, 0.5 mg of adenosylcobalamin, 5 mg magnesium, 10 mg of coenzyme Q10, and 2.1 mg of riboflavin.

Formula 16 was a dissolving tablet with 5.6 mg of cyanocobalamin, 0.5 mg of methylcobalamin, 0.5 mg of adenosylcobalamin, 10 mg of coenzyme Q10, and 1 mg of riboflavin.

Given that cobalamin is a coenzyme involved in the synthesis of lipids (fats), carbohydrates and proteins there are many possible biochemical pathways in which cobalamin may affect weight control. The following ingredients may be included in the current weight-control products, or be used as separate products in conjunction with the current product: methionine (one of the sulfur-containing amino acids, which acts as a lipotropic agent to prevent excess fat buildup in the liver and the body, is believed to be helpful in preventing fatigue and Methionine reacts with adenosine triphosphate (ATP) to form S-adenosyl methionine, a potent donor of methyl groups, choline (in the liver it plays a role in the processing and excretion of chemical waste products), inositol (a nutrient belonging to the B vitamin complex, closely associated with choline, it aids in the metabolism of fats and helps reduce blood cholesterol, and participates in action of serotonin, a neurotransmitter known to control mood and appetite), homocysteine (the conversion of homocysteine to methionine requires a cobalamin containing transmethylase), nitric oxide (buildup, squelching and reduction of NO), and methylmalonic acid (an increase in the urinary and serum concentrations of methylmalonic acid, which is an alternative product of a cobalamin dependant pathway within the mitochondria); pyrroloquinoline quinone (PQQ) which is believed to facilitate the proliferation of mitochondria, ascorbic acid, folic acid, niacinamide, omega-3 fatty acids (especially eicosapentaenoic acid, docosahexaenoic acid, and alpha-linolenic acid), palmitate, panthothenic acid, pyridoxine HCL, riboflavin, thiamine mononitrate, S-adenosyl methionine (SAMe), tocopheryl, tocopheryl acetate, vitamin D2, vitamin D3, hydroxycitric acid (garcinia cambogia or tamarind), acai, apple, blackberry, blueberry, chile peppers, cinnamon, coffee, green coffee, cranberry, tea, green tea, mushrooms, oatmeal, quinoa, raspberry, strawberry, and their respective metabolites, analogues, acids, hydroxyl groups, esters, salts, alcohols, acyl groups, extracts or a combination thereof; and others obvious to those familiar with the art.

In one preferred embodiment, the current invention can have pyrroloquinoline quinone (PQQ) included in the formula, or have PQQ administered separately, with the PQQ used once or twice or three times per day and with each tablet containing between 1 mg and 100 mg of PQQ.

The products above for normalizing body weight and body mass index (BMI) may be used alone, in addition to an exercise regimen, and/or in addition to a restricted-calorie diet regimen.

Other uses and preparations of the current invention, alone or in combination with other products not mentioned herein which are obvious to anyone skilled in the art, are also to be incorporated in the current invention.

### SUMMARY OF THE INVENTION

The present invention is directed at safe and effective orally-dissolving cobalamin tablets to support the normalization of weight and body mass indices (BMI) in underweight, overweight and obese individuals.

The inventor directed a phase III, placebo-controlled, nutritional study on 1,551 test subjects, and the surprising and unexpected study findings substantiate the invention.

A non-obvious regimin of 3.3 mg, twice-daily cobalamin tablets taken over a course of 42 consecutive days is disclosed, along with other similar regimins, is disclosed.

What I claim:

1. A method of normalizing body weight and body mass index in a human in need thereof, comprising placing in the mouth of said human, dissolving and swallowing a normalizing body weight and body mass index effective amount of normalizing body weight and body mass index product that is selected from the group consisting of cyanocobalamin, methylcobalamin, adenosylcobalamin, hydroxycobalamin, and combinations thereof.

2. The method of claim 1, wherein said normalizing body weight and body mass index product is administered to the mouth in a form selected from the group consisting of an orally-dissolving tablet, a dissolving strip, a lozenge, a spray, a tablet, a sublingual lozenge, a nasal spray, an injection, an ointment, a capsule, a dot, a solution, an emulsion, an encapsulated microsphere and a suspension.

3. The method of claim 1, wherein said administration of said normalizing body weight and body mass index product comprises repeated delivery ranging from about 5 days to about 90 days.

4. The method of claim 1, wherein said administration of said normalizing body weight and body mass index product comprises repeated delivery ranging from about once to three times daily.

5. The method of claim 1, wherein said normalizing body weight and body mass index product is ingested by being placed in the mouth, on top of the tongue, under the tongue and combinations thereof, and then swallowed.

6. The method of claim 1, wherein said cyanocobalamin is administered in one portion from about 0.05 mg to about 6 mg, said methylcobalamin is administered in one portion from about 0.05 mg to about 6 mg, and said adenosylcobalamin is administered in one portion from about 0.05 mg to about 6 mg.

7. The method of claim 1, wherein said amount of normalizing body weight and body mass index product that is selected from the group consisting of cyanocobalamin, methylcobalamin, adenosylcobalamin, hydroxycobalamin, and combinations thereof and is administered in one portion of about 3.3 mg.

8. The method of claim 1, wherein said normalizing body weight and body mass index product comprises selecting from said cyanocobalamin, said methylcobalamin, and said adenosylcobalamin, and said hydroxycobalamin the pharmaceutically-acceptable salts, chemical precursors, and metabolites thereof.

9. A composition for normalizing body weight and body mass index in a human in need thereof, comprising placing in the mouth of said human, dissolving and swallowing a normalizing body weight and body mass index product, the composition comprising from about 0.05 mg to about 6 mg cyanocobalamin; from about 0.05 mg to about 6 mg methylcobalamin; from about 0.05 mg to about 6 mg adenosylcobalamin; from about 3 mg to about 8 mg magnesium, from about 0.1 mg to about 250 mg L-carnitine, from about 5 mg to about 100 mg coenzyme Q10, and from about 0.1 mg to about 25 mg riboflavin.

10. The composition of claim 9, wherein said normalizing body weight and body mass index product is administered to the mouth in a form selected from the group consisting of an orally dissolving tablet, a dissolving strip, a lozenge, a spray, a tablet, a sublingual lozenge, a nasal spray, an injection, an ointment, a capsule, a dot, a solution, an emulsion, an encapsulated microsphere and a suspension.

11. The composition of claim 9, wherein said administration of said normalizing body weight and body mass index product comprises repeated delivery ranging from about 5 days to about 90 days.

12. The composition of claim 9, wherein said administration of said normalizing body weight and body mass index product comprises repeated delivery ranging from about once to three times daily.

13. A composition for normalizing body weight and body mass index in a human in need thereof, comprising placing in the mouth of said human, dissolving and swallowing a normalizing body weight and body mass index product, the composition comprising from about 2.5 mg to about 3.5 mg cyanocobalamin, from about 0.05 mg to about 0.3 mg methylcobalamin, from about 0.05 mg to about 0.05 mg adenosylcobalamin, from about 0.3 mg to about 500 mg magnesium, from about 0.01 mg to about 250 mg L-carnitine, from about 5 mg to about 500 mg coenzyme Q10, from about 0.1 mg to about 25 mg riboflavin, from about 100 mg to about 300 mg xlyitol, from about 50 mg to about 200 mg glycine, from about 10 mg to about 200 mg inositol, from about 10 mg to about 150 mg cherry flavor powder, from about 10 mg to about 2000 mg taurine, from about 1 mg to about 100 mg microcrystalline cellulose, from about 1 mg to about 25 mg sorbitol, from about 0.1 mg to about 5 mg Syloid 244, from about 0.1 mg to about 5 mg lauric acid, and combinations thereof.

14. The composition of claim 13, wherein said normalizing body weight and body mass index product is administered to the mouth in a form selected from the group consisting of an orally dissolving tablet, a dissolving strip, a lozenge, a spray, a tablet, a sublingual lozenge, a nasal spray, an injection, an ointment, a capsule, a dot, a solution, an emulsion, an encapsulated microsphere and a suspension.

15. The composition of claim 13, wherein said administration of said normalizing body weight and body mass index product comprises repeated delivery ranging from about 5 days to about 90 days.

16. The composition of claim 13, wherein said administration of said normalizing body weight and body mass index product comprises repeated delivery ranging from about once to three times daily.
